(19) United States
(12) Patent Application Publication
(10) Pub. No.: US 2024/0366369 A1
(43) Pub. Date: Nov. 7, 2024

(54) PROSTHETIC VALVE ACTUATOR ATTACHMENT STATUS DETECTION SYSTEM

(71) Applicant: EDWARDS LIFESCIENCES CORPORATION, Irvine, CA (US)

(72) Inventors: Joseph Mordechai Leichner, Caesarea (IL); Anatoly Dvorsky, Haifa (IL); Anatoly Dvorsky, Haifa (IL)

(21) Appl. No.: 18/775,235

(22) Filed: Jul. 17, 2024

Related U.S. Application Data
(63) Continuation of application No. PCT/US2023/010817, filed on Jan. 13, 2023.
(60) Provisional application No. 63/301,001, filed on Jan. 19, 2022.

(51) Int. Cl. A61F 2/24

(52) U.S. Cl. CPC A61F 2/2418; A61F 2/243; A61F 2/2436; A61F 2250/0096

(57) ABSTRACT

The present invention relates to systems, assemblies and methods for detecting attachment status of a prosthetic valve actuator. In an example, a prosthetic valve actuator attachment status detection system is constituted of: at least one actuator, a frame of the prosthetic valve movable by the at least one actuator between a radially compressed configuration and a radially expanded configuration, and at least one pressure sensor secured to the at least one actuator.

### CROSS-REFERENCE TO RELATED APPLICATIONS

This application is a continuation of International Application No. PCT/US2023/010817, filed Jan. 13, 2023, which claims benefit of U.S. Provisional Application No. 63/301,001, filed Jan. 19, 2022, the contents of each of which are herein incorporated by reference in their entirety.

### FIELD

The present invention relates to the field of prosthetic valves, and in particular to systems, assemblies and methods for detecting attachment status of a prosthetic valve actuator.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures is performed to repair or replace a heart valve. Surgeries are prone to an abundance of clinical complications, hence alternative less invasive techniques of delivering a prosthetic heart valve over a catheter and implanting it over the native malfunctioning valve, have been developed over the years.

Mechanically expandable valves are a category of prosthetic valves that rely on a mechanical actuation mechanism for expansion. The actuation mechanism usually includes a plurality of actuation/locking assemblies, releasably connected to respective actuation members of the valve delivery system, controlled via the handle for actuating the assemblies to expand the valve to a desired diameter. The assemblies may optionally lock the valve's position to prevent undesired recompression thereof, and disconnection of the delivery system's actuation member from the valve actuation/locking assemblies, to enable retrieval thereof once the valve is properly positioned at the desired site of implantation.

After implanting a prosthetic valve, such as a mechanically expandable valve, the actuation mechanism is detached from the prosthetic valve and extracted. If the actuation mechanism has not properly detached, the prosthetic valve will be extracted along with the actuation mechanism. What is desired, and not provided by the prior art, is a mechanism for checking that the actuation mechanism has properly detached.

Similarly, at certain stages in the lifetime of the prosthetic valve it is important to determine that the actuators are attached to the prosthetic valve, such as after manufacturing, during packaging, during transportation, while opening, while delivering through the patient's body, while maneuvering to position, and during deployment. What is desired, and not provided by the prior art, is a mechanism for checking that the actuation mechanism is properly attached.

### SUMMARY

The present disclosure is directed toward devices, assemblies and methods for detecting detachment of a prosthetic valve actuator. This is accomplished in some examples by providing a prosthetic valve actuator attachment status detection system, comprising: at least one actuator; a frame of the prosthetic valve movable by the at least one actuator between a radially compressed configuration and a radially expanded configuration; and at least one pressure sensor secured to the at least one actuator.

In some examples, the at least one pressure sensor is an optical pressure sensor. In some examples, the at least one pressure sensor is a fiber-optic pressure sensor.

In some examples, the at least one pressure sensor is embedded in the at least one actuator. In some examples, the at least one pressure sensor is positioned at an edge of the at least one actuator.

In some examples, the system further comprises a detection module configured to: receive an output of the at least one pressure sensor; based at least in part on the received output, determine the attachment status of the at least one actuator in relation to the frame; and based at least in part on the determination, output an indication of the determined attachment status.

In some examples, the determined attachment status comprises an indication of whether the at least one actuator has detached from the frame. In some examples, the determined attachment status is an indication of whether the at least one actuator is attached to the frame.

In some examples, the determined attachment status is an indication of whether the at least one actuator is correctly attached to the frame. In some examples, the detection module is further configured to compare the received output of the at least one pressure sensor to a predetermined pressure value, wherein the determination of the attachment status is based at least in part on an outcome of the comparison.

In some examples, the frame comprises: at least one axially extending post, each of the at least one axially extending post comprising a proximal member and a distal member that are axially movable relative to one another to permit the frame to radially expand and/or compress, at least one rod, each associated with a respective one of the at least one axially extending post and rotatably coupled to the proximal member and distal member thereof, wherein a rotation of the at least one rod radially expands and/or radially compresses the frame, the rotation of the at least one rod performed by the at least one actuator, and wherein the determination of the attachment status of the at least one actuator comprises a determination of whether the at least one actuator is attached to the at least one rod.

In some examples, wherein each of the proximal member and distal member of each of the at least one axially extending post comprises an inner bore, the respective one of the at least one rod extending through the inner bores of the proximal member and distal member.

In some examples, each of the at least one actuator comprises: a pair of elongated members; and a movable sleeve configured to compress the pair of elongated members such that the compressed pair of elongated members are fastened to a respective one of the at least one rod, wherein when the movable sleeve is moved away from the pair of elongated members the pair of elongated members detach from the respective rod, the determination of the attachment status of the at least one actuator comprising a determination whether the pair of elongated members have detached from the respective rod.

In some examples, each of the at least one actuator comprises: a pair of elongated members; and a movable sleeve configured to compress the pair of elongated members such that the compressed pair of elongated members are fastened to a respective one of the at least one rod, wherein when the movable sleeve is moved away from the pair of elongated members the pair of elongated members detach from the respective rod, the determination of the attachment status of the at least one actuator comprising a determination whether the pair of elongated members are attached to the respective rod.

In some examples, the at least one axially extending post comprises a plurality of axially extending posts and the at least one rod comprises a plurality of rods, wherein the at least one actuator comprises a plurality of actuators, the rotation of each of the plurality of rods performed by a respective one of the plurality of actuators, and wherein the determination of the attachment status of the at least one actuator comprises a determination of the attachment status of each of the plurality of actuators.

In some examples, the determination of the attachment status of the at least one actuator comprises a determination whether each of the plurality of actuators has detached from the respective rod.

In some examples, the determination of the attachment status of the at least one actuator comprises a determination whether each of the plurality of actuators is attached to the respective rod.

In some examples, the determination of the attachment status of the at least one actuator comprises a determination whether each of the plurality of actuators is correctly attached to the respective rod.

Certain examples of the present invention may include some, all, or none of the above advantages. Further advantages may be readily apparent to those skilled in the art from the figures, descriptions, and claims included herein. Aspects and examples of the invention are further described in the specification herein below and in the appended claims.

Unless otherwise defined, all technical and scientific terms used herein have the same meaning as commonly understood by one of ordinary skill in the art to which this invention pertains. In case of conflict, the patent specification, including definitions, governs. As used herein, the indefinite articles “a” and “an” mean “at least one” or “one or more” unless the context clearly dictates otherwise.

The following examples and aspects thereof are described and illustrated in conjunction with systems, tools and methods which are meant to be exemplary and illustrative, but not limiting in scope. In various examples, one or more of the above-described problems have been reduced or eliminated, while other examples are directed to other advantages or improvements.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A illustrates a high-level perspective view of a first example of a prosthetic valve, according to some examples;

FIG. 1B illustrates a high-level perspective view of a portion of the prosthetic valve of FIG. 1A, according to some examples;

FIG. 1C illustrates a high-level perspective view of a portion of a second example of a prosthetic valve, according to some examples;

FIG. 2 illustrates a high-level side view of the prosthetic valve of FIG. 1A attached to a delivery system, according to some examples;

FIGS. 3A-3B illustrate various high-level views of a distal portion of an actuator of the prosthetic valve of FIG. 1A;

FIGS. 4A-4C illustrate various steps in the detachment of an actuator from the prosthetic valve of FIG. 1A, according to some examples; and

FIG. 5 illustrates a high-level perspective view of a case for storing the prosthetic valve of FIG. 1A, according to some examples.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

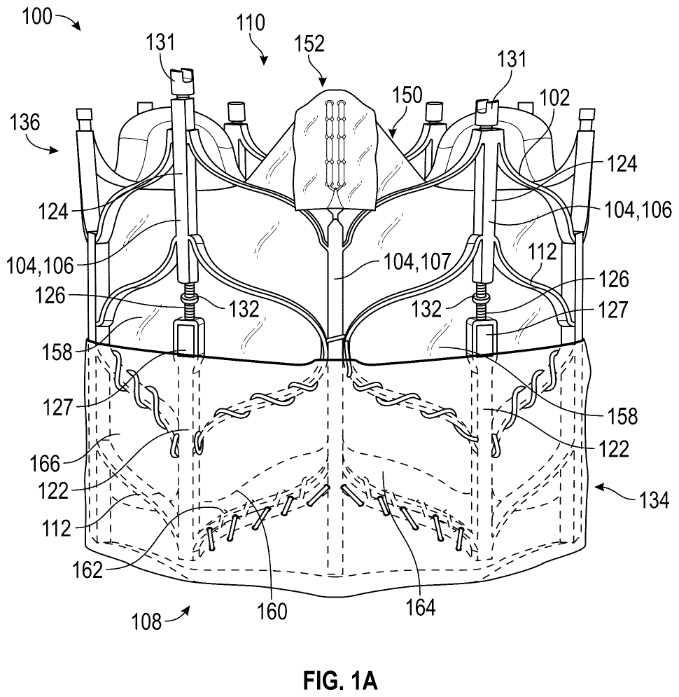

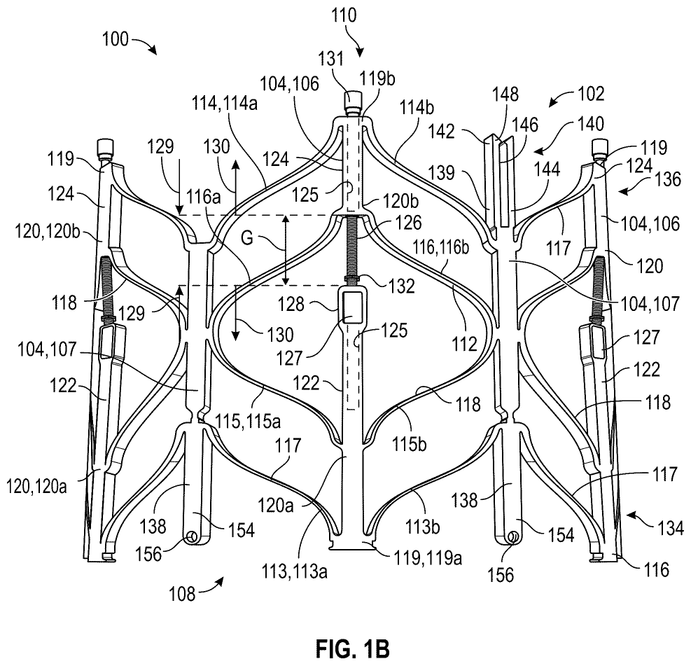

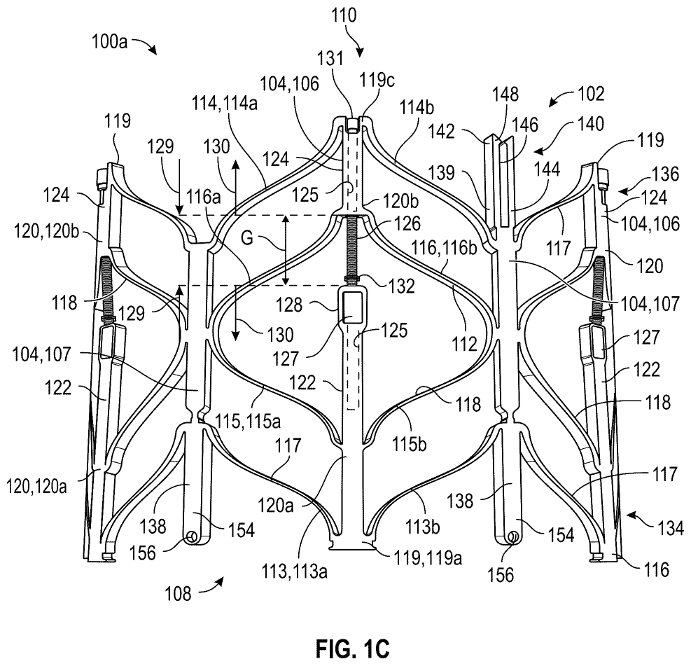

FIG. 1A illustrates a high-level perspective view of a prosthetic valve 100 and FIG. 1B illustrates a high-level perspective view of a portion of prosthetic valve 100, FIGS. 1A-1B being described together. Prosthetic valve 100 comprises a frame 102, a portion of which is illustrated in FIG. 1B. In FIG. 1A, frame 102 is illustrated with an optional valvular structure (e.g., leaflets) and an optional skirt assembly, as will be described below. While only one side of frame 102 is depicted in FIG. 1B, it should be appreciated that frame 102 forms an annular structure having an opposite side that is substantially identical to the portion shown in FIG. 1B, as illustrated in FIG. 1A.

The term “prosthetic valve”, as used herein, refers to any type of a prosthetic valve deliverable to a patient's target site over a catheter, which is radially expandable and compressible between a radially compressed, or crimped, state, and a radially expanded state. Thus, a prosthetic valve 100 is crimped or retained by a delivery apparatus in a compressed state during delivery, and then expanded to the expanded state once prosthetic valve 100 reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state.

Frame 102 comprises a plurality of axially extending posts 104. In some examples, one or more posts 104 are configured as radial adjustment mechanisms 106. In some examples, one or more posts 104 are configured as support posts 107. In some examples, one or more posts 104 are configured as radial adjustment mechanism 106 and one or more posts 104 are configured as support posts 107. In some examples, radial adjustment mechanisms 106 are integrated into frame 102, thereby reducing the crimp profile and/or bulk of prosthetic valve 100. Integrating radial adjustment mechanisms 106 into frame 102 also simplifies the design of prosthetic valve 100, making the prosthetic valve 100 cheaper and/or easier to manufacture. Radial adjustment mechanism 106 is used to radially expand and/or radially compress frame 102, as will be described below.

Posts 104 extend axially between a distal end 108 and a proximal end 110 of frame 102. In some examples, such as the example shown in FIGS. 1A-1B, distal end 108 is an inflow end and proximal end 110 is an outflow end, such as when prosthetic valve 100 is configured to replace a native aortic valve and prosthetic valve 100 is delivered to the native aortic valve via a retrograde, transfemoral delivery approach (e.g., through a femoral artery and the aorta). However, in other examples, distal end 108 is the outflow end and proximal end 110 is the inflow end, such as when prosthetic valve 100 is delivered to the native aortic valve via a transapical delivery approach, or when the prosthetic valve is configured to replace a native mitral valve and is delivered to the native mitral valve in a trans-septal delivery approach in which the delivery apparatus and the prosthetic valve are advanced into the right atrium, through the atrial septum, and into the left atrium, wherein the right atrium may be accessed via a femoral vein and inferior vena cava or via the superior vena cava.

Posts 104 are coupled together by a plurality of circumferentially extending link members or struts 112. Each strut 112 extends circumferentially between adjacent posts 104 to connect all of the axially extending posts 104. In some examples, frame 102 comprises an equal number of support posts 107 and radial adjustment mechanisms 106. In one further example, radial adjustment mechanisms 106 and support posts 107 are arranged in an alternating order such that each strut 112 is positioned between a radial adjustment mechanism and a support post 107, i.e., each strut 112 is coupled on one end to a post 104 that is configured as a radial adjustment mechanism 106 and is coupled on the other end to a post 104 that is configured as a support post 107. However, this is not meant to be limiting in any way, and frame 102 can include different numbers of support posts 107 and radial adjustment mechanisms 106, without exceeding the scope of the disclosure. Similarly, radial adjustment mechanisms 106 and support posts 107 can be arranged in a non-alternating order, without exceeding the scope of the disclosure.

In some examples, as illustrated in FIG. 1B, struts 112 include: a first row of struts 113 at or near distal end 108 of frame 102; a second row of struts 114 at or near proximal end 110 of frame 102; and third and fourth rows of struts 115, 116, respectively, positioned axially between the first and second rows of struts 113, 114. In some examples, struts 112 define a plurality of cells (i.e., openings) in frame 102. For example, struts 113, 114, 115, and 116 at least partially form define a plurality of first cells 117 and a plurality of second cells 118. Specifically, each first cell 117 is formed by two struts 113a, 113b of the first row of struts 113, two struts 114a, 114b of the second row of struts 114, and two of the posts 104. Each second cell 118 is formed by two struts 115a, 115b of the third row of struts 115 and two struts 116a, 116b of the fourth row of struts 116. As illustrated in FIG. 1B, each second cell 118 is disposed within one of first cells 117.

In some examples, as illustrated in FIG. 1B, struts 112 of frame 102 comprise a curved shape. In some examples, each first cell 117 has an axially-extending elliptical shape including first and second apices 119 (e.g., proximal apex 119a and distal apex 119b) disposed at the major vertices of the ellipse. In some examples, each second cell 118 has a circumferentially-extending elliptical shape including first and second apices 120 (e.g., proximal apex 120a and distal apex 120b) disposed at the minor vertices of the ellipse. In some examples, frame 102 comprises six first cells 117 arranged circumferentially in a row, six second cells 118 arranged circumferentially in a row within the six first cells 117, and thirteen posts 104. However, this is not meant to be limiting in any way, and frame 102 can comprise a greater or fewer number of first cells 117 and a correspondingly greater or fewer number of second cells 118 and posts 104, without exceeding the scope of the disclosure.

In some examples, some of posts 104, such as the posts 104 that are configured as radial adjustment mechanisms 106, are discontinuous and each include a distal member 122 and a proximal member 124 that are axially separated from one another by a gap G (FIG. 1B). Proximal member 124 (i.e., the upper post shown in FIG. 1B) extends axially from proximal end 110 of frame 102 towards the respective distal member 122, and distal member 122 (i.e., the lower post shown in FIG. 1B) extends axially from proximal end 110 of frame 102 towards the respective proximal member 124. In some examples, each distal member 122 is axially aligned with a corresponding proximal member 124 and one or both of the members 122, 124 include an inner bore 125, so that, for example, the axially aligned pair of members 122, 124 can receive an actuation member, such as in the form of a substantially straight threaded rod 126 as shown in the illustrated example.

Threaded rod 126 is coupled to the respective proximal member 124 and/or distal member 122. In some examples, rod 126 is rotatably coupled to proximal member 124 such that rod 126 can only be rotated to move rod 126 axially relative to proximal member 124 but otherwise cannot move relative to proximal member 124 (e.g., it cannot slide axially relative to proximal member 124 or move circumferentially and/or radially relative to proximal member 124). Similarly, in some examples, rod 126 is restrained radially and/or circumferentially by distal member 122 such that rod 126 can only be rotated and/or moved axially relative to distal member 122 but otherwise cannot move relative to the distal member 122 (e.g., radially and/or circumferentially). In some examples, threaded rod 126 is inserted through an inner bore 125 of one of the proximal members 124 and into a stationary nut 127 and/or bore 125 included in the respective distal member 122. In other examples, threaded rod 126 does extend through a bore in proximal member 124 and instead is secured to proximal member 124 using other suitable structures such as guides, straps, loops, collars, etc.

In some examples, stationary nut 127 is included at a proximal end portion 128 of distal member 122. In some examples, threaded rod 126 extends distally past nut 127 into inner bore 125 of distal member 122. Stationary nut 127 is held in a fixed position relative to distal member 122 such that nut 127 does not rotate relative to distal member 122. In this way, whenever threaded rod 126 is rotated (e.g., by a physician), threaded rod 126 rotates relative to both stationary nut 127 and distal member 122. In some examples, nut 127 includes a threaded bore that that is configured to engage the threads of threaded rod 126 to prevent rod 126 from moving axially relative to nut 127 and distal member 122 unless threaded rod 126 is being rotated. In other examples, in lieu of using nut 127, at least a portion of inner bore 125 of distal member 122 is threaded. For example, proximal end portion 128 of distal member 122 can comprise inner threads configured to engage threaded rod 126 such that rotation of threaded rod 126 causes it to move axially.

When threaded rod 126 is inserted into and/or otherwise coupled to a pair of axially aligned members 122, 124, the pair of axially aligned members served to expand and/or compress frame 102 when threaded rod 126 is rotated, as described below. In some examples, respective threaded rods 126 are inserted through each pair of axially aligned members 122, 124 so that all of the members 122, 124 serve to expand and/or compress frame 102. In some examples, frame 102 comprises six pairs of members 122, 124, and each of the six pairs of members 122, 124 is has a respective threaded rod 126, such that the expansion and/or compression of frame 102 is controlled at six different points. In other examples (not shown), not all pairs of members 122, 124 have a respective threaded rod 126.

The threaded rod 126 is rotated relative to the respective nut 127 and/or the respective member 122 to axially foreshorten and/or axially elongate frame 102, thereby radially expanding and/or radially compressing, respectively, frame 102. Specifically, when threaded rod 126 is rotated relative to nut 127 and/or member 122, the respective members 122, 124 can move axially relative to one another, thereby widening or narrowing the gap G (FIG. 1B) separating members 122 and 124, and thus radially compressing or radially expanding the prosthetic valve 100, respectively. Thus, the gap G (FIG. 1B) between members 122 and 124 narrows as frame 102 is radially expanded and widens as frame 102 is radially compressed. Rotation of threaded rod 126 in a first direction (e.g., clockwise) causes corresponding axial movement of members 122 and 124 toward one another (as shown by arrows 129 in FIG. 1B), thereby radially expanding frame 102, while rotation of the threaded rod 126 in an opposite second direction (e.g., counterclockwise) causes corresponding axial movement of members 122 and 124 away from one another (as shown by arrows 130 in FIG. 1B), thereby radially compressing frame 102.

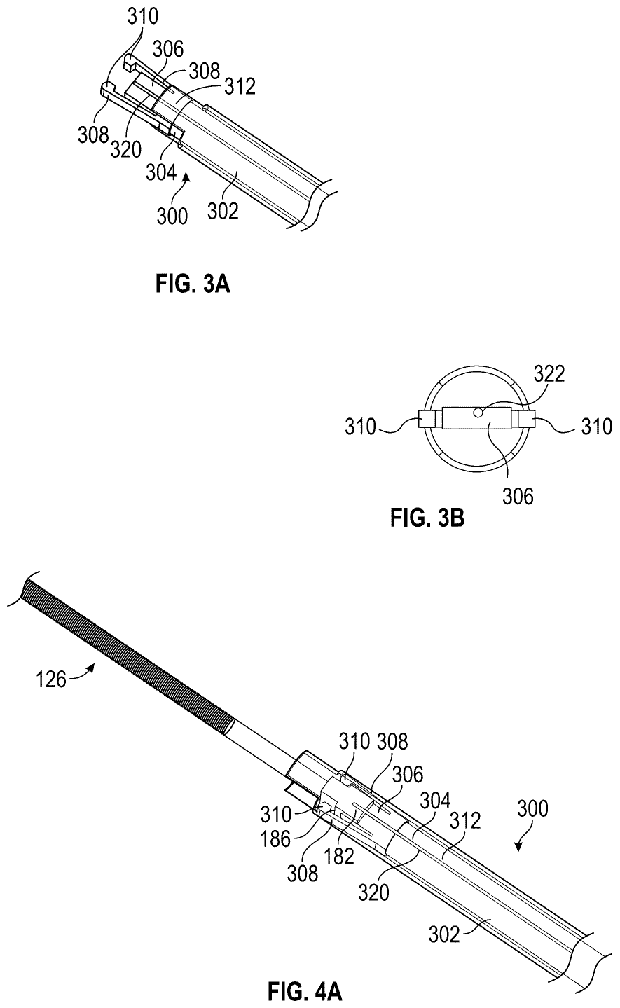

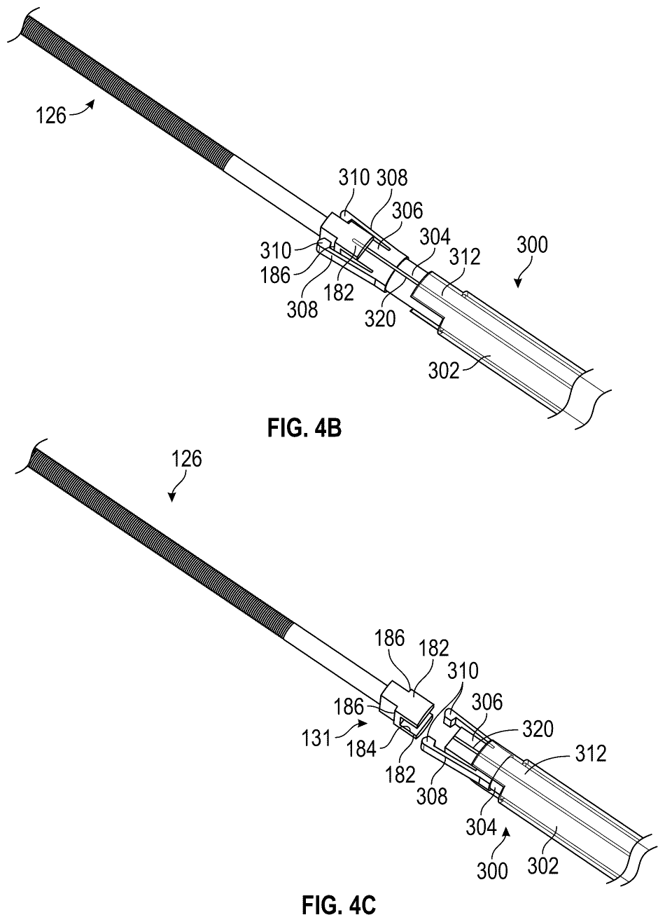

In some examples, threaded rod 126 extends proximally past the proximal end of proximal member 124 and in one further example includes a head portion 131 at its proximal end. Head portion 131 serves at least two functions. First, as will be described in greater detail below with reference to FIGS. 3-4, head portion 131 couples threaded rod 126 to a respective actuator that is used to radially expand and/or radially compress frame 102. Second, head portion 131 prevents proximal member 124 from moving proximally relative to threaded rod 126 and can apply a distally directed force to proximal member 124, such as when radially expanding frame 102. Specifically, in some examples, head portion 131 has a width greater than a diameter of inner bore 125 of the respective proximal member 124 such that head portion 131 is prevented from moving into inner bore 125. Thus, as threaded rod 126 is threaded farther into nut 127, head portion 131 draws closer to nut 127 and distal member 122, thereby drawing proximal member 124 towards distal member 122, and thereby axially foreshortening and radially expanding frame 102.

In some examples, each threaded rod 126 comprises a stopper 132 (e.g., a nut) disposed thereon. Each stopper 132 is disposed on the respective threaded rod 126 such that it sits within gap G. Furthermore, stopper 132 is integrally formed on or fixedly coupled to threaded rod 126 such that it does not move relative to threaded rod 126. Thus, stopper 132 remains in a fixed position on the threaded rod 126 such that it moves together with threaded rod 126. Stopper 132 thus limits the distance of movement for threaded rod 126 in each direction to predetermined values.

As described above, in some examples, a subset of posts 104 are configured as support posts 107. In some examples, support posts 107 extend axially between distal and proximal end portions 134, 136 of frame 102. In some examples, some, or all of support posts 107 extend between a distal end portion 138 and a proximal end portion 139. Proximal end portion 139 of one or more support posts 107 include a commissure support member 140. In some examples, commissure support member 140 comprises first and second commissure arms 142, 144 defining a commissure opening 146 therebetween. In some examples, proximal end of each commissure arm 142, 144 comprises a tooth 148 extending into commissure opening 146.

In some examples, commissure opening 146 extends through a thickness of the respective post 107 and is configured to accept a portion of a valvular structure 150 (e.g., a commissure 152) to couple the valvular structure 150 to frame 102. For example, each commissure 152 is mounted to a respective commissure support member 140, such as by inserting a pair of commissure tabs of adjacent leaflets through opening 146 and suturing the commissure tabs to each other and/or to arms 142, 144. In some examples, opening 146 is fully enclosed by post 107 (e.g., not extending to the proximal edge) such that a portion of valvular structure 150 is slid radially (rather than axially) into the commissure opening 146. Teeth 148 can help retain commissure 152 within commissure opening 146. In the illustrated example, commissure opening 146 has a substantially rectangular shape and extends to the distal end of the respective post 107, however this is not meant to be limiting in any way. In other examples, the commissure opening can have any of a variety of shapes (e.g., square, oval, square-oval, triangular, L-shaped, T-shaped, C-shaped, etc.), without exceeding the scope of the disclosure.

Though only one support post 107 comprising a commissure support member 140 is shown in FIG. 1B, it should be noted that frame 102 can comprise any number of support posts 107, any number of which comprising commissure support members 140. For example, frame 102 can comprise six support posts 107, three of which comprising commissure support members 140, as illustrated. However, in other examples, frame 102 can comprise more or less than six support posts 107 and/or more or less than three commissure support members 140.

In some examples, distal end portion 138 of each support post 107 comprises an extension 154 that extends toward the distal end 108 of frame 102. In some examples, extension 154 comprises an aperture 156 extending radially through a thickness of the respective extension 154. In some examples, extension 154 extends such that a distal edge of extension 154 aligns with or substantially aligns with distal end 108 of the frame 102. During use, extension 154 can prevent or mitigate portions of an outer skirt from extending radially inwardly and thereby prevent or mitigate obstruction of flow through distal end 108 of frame 102 caused by the outer skirt. Extensions 154 can further serve as supports to which portions of the inner and/or outer skirts is coupled. For example, sutures used to connect the inner and/or outer skirts is wrapped around the extensions 154 and/or can extend through openings 156.

In some examples, frame 102 is a unitary and/or fastener-free frame that is constructed from a single piece of material (e.g., Nitinol). In some examples, the plurality of cells 117, 118 are formed by removing portions (e.g., via laser cutting) of the single piece of material. Threaded rods 126 can then be inserted through the bores 125 in proximal members 124 and threaded into threaded nuts 127.

As illustrated in FIG. 1A, prosthetic valve 100 further comprises valvular structure 150, which is coupled to and supported inside frame 102. Valvular structure 150 is configured to regulate the flow of blood through prosthetic valve 100, from the inflow end to the outflow end. In some examples, valvular structure 150 includes a leaflet assembly comprising one or more leaflets 158 made of flexible material. Leaflets 158 are made from in whole or part, biological material, bio-compatible synthetic materials, or other such materials. Suitable biological material can include, for example, bovine pericardium (or pericardium from other sources). Leaflets 158 are secured to one another at their adjacent sides to form commissures 152, each of which is secured to a respective post 104 (e.g., to a support post 107) and/or to other portions of frame 102.

In the example illustrated in FIG. 1A, valvular structure 150 includes three leaflets 158, which are arranged to collapse in a tricuspid arrangement. Each leaflet 158 has a proximal edge portion 160. As shown in FIG. 1A, in some examples, proximal edge portions 160 of leaflets 158 define an undulating, curved scallop edge that follows or tracks portions of struts 112 of frame 102 in a circumferential direction when frame 102 is in a radially expanded configuration. The proximal edges 160 of leaflets is referred to as a “scallop line.”

In some examples, as shown in FIG. 1A, proximal edge portions 160 of leaflets 158 are sutured to an inner skirt 164 generally along the scallop line. Inner skirt 164 can in turn be sutured, via one or more sutures 162, to adjacent struts 112 of frame 102. In other examples, leaflets 158 are sutured directly to frame 102 along the scallop line. Inner skirt 164 can function as a sealing member to prevent or decrease perivalvular leakage, to anchor leaflets 158 to frame 102, and/or to protect leaflets 158 against damage caused by contact with frame 102 during crimping and during working cycles of prosthetic valve 100.

In some examples, prosthetic valve 100 further comprises an outer skirt 166 mounted on an outer surface of frame 102. Outer skirt 166 can function as a sealing member for prosthetic valve 100 by sealing against the tissue of the native valve annulus and helping to reduce paravalvular leakage past prosthetic valve 100. Inner and outer skirts 164, 166 are formed from any of various suitable biocompatible materials, including any of various synthetic materials, including fabrics (e.g., polyethylene terephthalate fabric) or natural tissue (e.g., pericardial tissue).

FIG. 1C illustrates a portion of a prosthetic valve 100a. Prosthetic valve 100a is in all respects similar to prosthetic valve 100, with the exception that struts 114a and 114b extend proximally along head portions 131 into apex 119c.

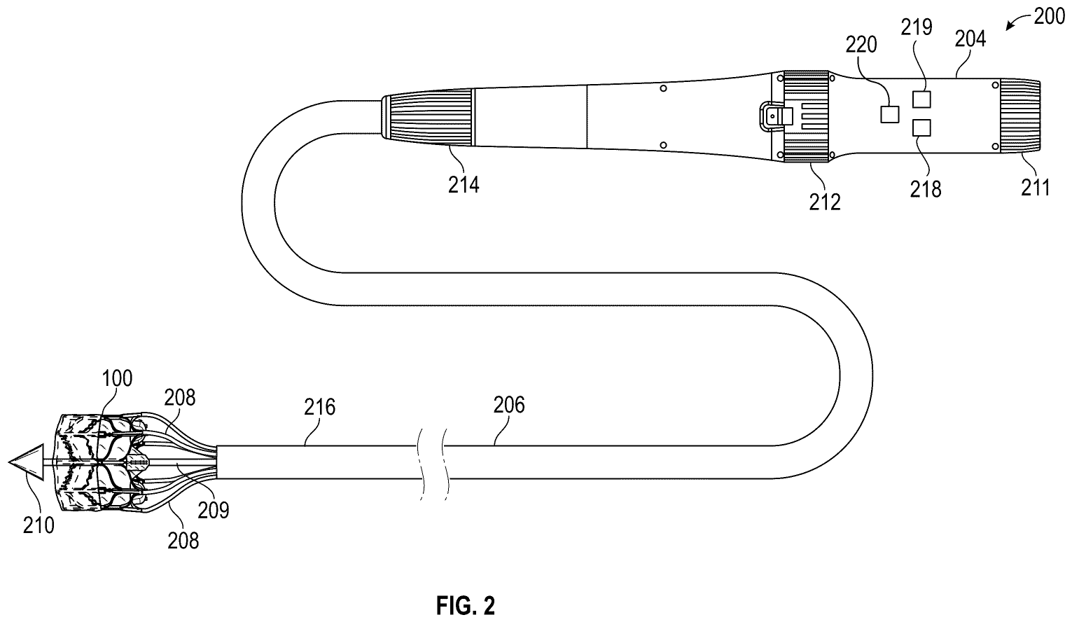

As described above, threaded rods 126 are removably coupled to an actuator assembly of a delivery apparatus. FIG. 2 illustrates a high-level side view of prosthetic valve 100 secured to a delivery apparatus 200 for delivering prosthetic valve 100 to a desired implantation location. It should be understood that delivery apparatus 200 and other delivery apparatuses disclosed herein can be used to implant prosthetic devices other than prosthetic valves, such as stents or grafts.

In the illustrated example, delivery apparatus 200 comprises: a handle 204; a first elongated shaft 206 (which comprises an delivery shaft in the illustrated example) extending distally from the handle 204; at least one actuator 208 extending distally through the delivery shaft 206; an elongated nosecone shaft 209 (which comprises an inner shaft in the illustrated example) extending through first shaft 206; and a nosecone 210 coupled to a distal end portion of nosecone shaft 209. In some examples, nosecone shaft 209 and nosecone 210 define a guidewire lumen for advancing prosthetic valve 100 through a patient's vasculature over a guidewire. Actuator assembly 208 is configured to radially expand and/or radially collapse frame 102 when actuated, such as by one or more knobs 211, 212 and 214 included on handle 204 of delivery apparatus 200. Though the illustrated example shows six actuators 208, this is not meant to be limiting in any way. In some examples, a respective actuator 208 is provided for each threaded rod 126 provided in frame 102.

In some examples, a distal end portion 216 of shaft 206 is sized to house prosthetic valve 100 in its radially compressed, delivery state during delivery thereof through the patient's vasculature. In this manner, distal end portion 216 of shaft 206 functions as a delivery sheath or capsule for prosthetic valve 100 during delivery.

The term “proximal”, as used herein, generally refers to the side or end of any device or a component of a device, which is closer to handle 204 or an operator of handle 204 when in use.

The term “distal”, as used herein, generally refers to the side or end of any device or a component of a device, which is farther from handle 204 or an operator of handle 204 when in use.

As will be described below, actuators 208 are releasably coupled to frame 102. Particularly, when actuated, actuator 208 transmits pushing and/or pulling forces from handle 204 to portions of frame 102 to radially expand and collapse the prosthetic valve as previously described. In some examples, actuators 208 are at least partially disposed within, and extend axially through, one or more lumens of delivery shaft 206. For example, actuators 208 can extend through a central lumen of the shaft 206 or through separate respective lumens formed in the shaft 206.

In some examples, the first knob 211 is a rotatable knob configured to produce axial movement of delivery shaft 206 relative to prosthetic valve 100 in the distal and/or proximal directions in order to deploy prosthetic valve 100 from delivery shaft 206 once prosthetic valve 100 has been advanced to a location at or adjacent to the desired implantation location with the patient's body. For example, rotation of the first knob 211 in a first direction (e.g., clockwise) retracts distal portion 216 of delivery shaft 206 proximally relative to prosthetic valve 100 and rotation of the first knob 211 in a second direction (e.g., counter-clockwise) advances distal portion 216 distally. In other examples, the first knob 211 can be actuated by sliding or moving knob 211 axially, such as pulling and/or pushing the knob. In other examples, actuation of first knob 211 (rotation or sliding movement of the knob 211) produce axial movement of actuators 208 (and therefore prosthetic valve 100) relative to distal portion 216 of delivery shaft 206 to distally advance prosthetic valve 100.

In some examples, second knob 212 is a rotatable knob configured to produce radial expansion and/or compression of frame 102. For example, rotation of second knob 212 can rotate threaded rods 126 of frame 102 via actuators 208, as will be described below. Rotation of second knob 212 in a first direction (e.g., clockwise) radially expands frame 102 and rotation of the second knob 212 in a second direction (e.g., counter-clockwise) radially collapses frame 102. In other examples, second knob 212 is actuated by sliding or moving knob 212 axially, such as pulling and/or pushing the knob.

In some examples, third knob 214 is a rotatable knob operatively coupled to a proximal end portion of each actuator 208. In some examples, third knob 214 is configured to retract an outer sleeve or support tube of each actuator 208 to disconnect actuators 208 from threaded rods 126 of frame 102, as further described below. Once actuators 208 are uncoupled from frame 102, delivery apparatus 200 can be removed from the patient, leaving just prosthetic valve 100 in the patient.

In some examples, delivery apparatus 200 further comprises: a detection module 218; and an indicator 219. In some examples, an optical system 220 is provided, as will be described below. In some examples (not shown), a communication component is further provided for communication with external systems.

In some examples (not shown), a memory is further provided and is configured to store signals received by detection module 218. A memory may include a suitable memory chip or storage medium such as, for example, a PROM, EPROM, EEPROM, ROM, flash memory, solid state memory, or the like.

In some examples, detection module 218 is implemented as one or more of: a dedicated circuitry; a micro-controller; a processor; an application-specific integrated circuit (ASIC); a field-programmable-gate-array (FPGA); or software running on a computing device. In an example where detection module 218 is implemented as software, a memory (not shown) is further provided, the memory having stored therein instructions which when run by one or more processors cause the one or more processors to run the software. In some examples, indicator 219 comprises a display.

FIG. 3A illustrates a high-level perspective view of a distal end 300 of an actuator 208 and FIG. 3B illustrates a high-level front view of distal end 300 of actuator 208. Distal end 300 is shown partially transparent in FIG. 3A for purposes of illustration. In some examples, distal end 300 of actuator 208 comprises: a sleeve 302; a driver 304; a central protrusion 306; a pair of elongated members 308, each comprising a protrusion, or tooth, at a distal end 310 thereof; a sensor 320. In some examples, central protrusion 306 extends distally from driver 304. In some examples, elongated members 308 extend distally from the sides of central protrusion 306, such that distal ends 310 are positioned distally to central protrusion 306. In some examples, as illustrated in FIG. 3B, sensor 320 is embedded within central protrusion 306. In some examples (not shown), sensor 320 is disposed on an outer surface of central protrusion 306.

In some examples, sensor 320 is implemented as a pressure sensor. In some examples, sensor 320 is implemented as an optical pressure sensor. In some examples, sensor 320 is implemented as a fiber-optic pressure sensor, comprising one or more optical fibers and a reflective membrane 322 at a distal end thereof. In one further example, as described above, an optical system 220 is provided within handle 204, the optical system comprising a light source and a light detector. In such an example, optical system 220 forms a portion of sensor 320. In some examples, the light source comprises a light-emitting diode (LED) and the light detector comprises a photodiode. In some examples, optical system 220 is in communication with detection module 218.

FIGS. 4A-4C illustrate various steps in the detachment of an actuator 208 from a respective threaded rod 126. Particularly, in FIGS. 4A-4C, distal portion 300 of an actuator 208 and a proximal portion of a threaded rod 126 are shown. In some examples, head portion 131 comprises first and second protrusions 182 defining a channel or slot 184 between protrusions 182, and one or more shoulders 186.

As illustrated in FIG. 4A, central protrusion 306 is situated within slot 184 of head portion 131 and distal ends 310 of elongated members 308 are secured distally to head portion 131 by sleeve 302. Particularly, in some examples, distal ends 310 are secured against shoulders 186 of head portion 131. Sleeve 302 is shown transparently for purposes of illustration. As illustrated, sleeve 302 compresses elongated members 308 such that they are secured to head portion 131. Thus, in this position, driver 304 can be rotated, as described above, thereby rotating head portion 131 and expanding/compressing prosthetic valve 100.

In some examples, central protrusion 306 is sized and shaped such that it is spaced apart from the inner walls of outer sleeve 302, such that the central protrusion 306 does not frictionally contact outer sleeve 302 during rotation. Although in the illustrated example central protrusion 306 has a substantially rectangular shape in its cross-section, this is not meant to be limiting in any way, and protrusion 306 can have any of various shapes, for example, square, triangular, oval, etc, without exceeding the scope of the disclosure.

In some examples, the distal end portion of sleeve 302 comprises a pair of support extensions 312 extending distally therefrom. In one further example, support extensions 312 are oriented such that, when central protrusion is inserted within head portion 131, support extensions 312 extend partially over a proximal end portion (e.g., the upper end portion) of proximal members 124 (not shown). The engagement of support extensions 312 with proximal member 124 in this manner can counter-act rotational forces applied to the frame by threaded rods 126 during expansion of prosthetic valve 100. In the absence of a counter-force acting against these rotational forces, the frame can tend to “jerk” or rock in the direction of rotation of the rods when they are actuated to expand the frame. The illustrated configuration is advantageous in that sleeves 302, when engaging proximal members 124 of the frame, can prevent or mitigate such jerking or rocking motion of the frame.

In some examples, sleeve 302 is advanced (moved distally) and/or retracted (moved proximally) relative to driver 304 via a control mechanism (e.g., knob 214) on handle 204 of delivery apparatus 200, by an electric motor, and/or by another suitable actuation mechanism. For example, the physician can turn knob 214 in a first direction to apply a distally directed force to sleeve 302 and can turn knob 214 in an opposite second direction to apply a proximally directed force to sleeve 302. Thus, when sleeve 302 does not abut prosthetic valve 100a and the physician rotates knob 214 in the first direction, sleeve 302 can move distally relative to driver 304, thereby advancing the sleeve 302 over driver 304 and elongated members 308. When sleeve 302 does abut prosthetic valve 100, the physician can rotate knob 214 in the first direction to push the entire prosthetic valve 100 distally via sleeve 302. Furthermore, when the physician rotates knob 214 in the second direction, sleeve 302 can move proximally relative to driver 304, thereby withdrawing/retracting sleeve 302 from the driver 304.

When expansion of prosthetic valve 100a is completed, sleeve 302 is retracted proximally off of elongated members 308, thus releasing elongated members 308, as described above and illustrated in FIG. 4B. Particularly, elongated members 308 are flexible, such that when not compressed by sleeve 302 they bend outwards, away from head portion 131. Actuator 208 is then retracted, as illustrated in FIG. 4C.

Although the above has been described in relation to an example where distal end 300 of actuator 208 comprises a central protrusion 306, which is inserted within a respective slit of head portion 131, this is not meant to be limiting in any way, and actuator 208 can connect to head portion 131 in other suitable configurations, without exceeding the scope of the disclosure.

Detection module 218 receives an output of sensor 320. In an example where sensor 320 is implemented as a pressure sensor, detection module 218 receives pressure values from sensor 320. Particularly, in such an example, the pressure applied to distal end 322 of sensor 320 is detected. In an example where sensor 320 is implemented an optical pressure sensor, the intensity and/or phase shift of the light reflected off distal end 322 of sensor 320 indicates the pressure applied thereto.

In some examples, based at least in part on the received pressure values, detection module 218 determines the attachment status of the respective actuator 208. In some examples, the attachment status is a determination of whether actuator 208 has detached from frame 102. In one further example, detachment from frame 102 is determined by detecting a drop in sensed pressure, since the pressure applied to distal end 322 of sensor 320 is no longer the pressure generated by actuator 208 being pressed against threaded rod 126. Rather, once being released, the pressure sensed by sensor 320 is the blood pressure flowing through the patient artery. In one further example, detection module 218 outputs an indication of the determination of whether actuator 208 has detached. In some examples, the drop in sensed pressure is compared to a respective predetermined pressure drop value. If the drop in sensed pressure is not greater than the predetermined pressure drop value, detection module 218 determines that actuator 208 has not fully detached. If, on the other hand, the drop in sensed pressure is greater than the predetermined pressure drop value, detection module 218 determines that actuator 208 has fully detached. In one further example, detection module 218 outputs an indication of the determination of whether actuator 208 has fully detached.

In some examples, the attachment status is a determination of whether actuator 208 is attached to frame 102. In one further example, attachment to frame 102 is determined by comparing the output of sensor 320 to a predetermined pressure value. If the output is less than the predetermined pressure value it is determined that actuator 208 is not attached to frame 102. In some examples, the attachment status is a determination of whether actuator 208 is correctly attached to frame 102. In such an example, the predetermined pressure value provides an indication that actuator 208 is fully attached to frame 102. In some examples, detection module 218 outputs an indication of the determination of whether actuator 208 is fully attached.

In some examples, indications output by detection module 218 are output at indicator 219. In some examples, indications output by detection module 218 are output to an external system.

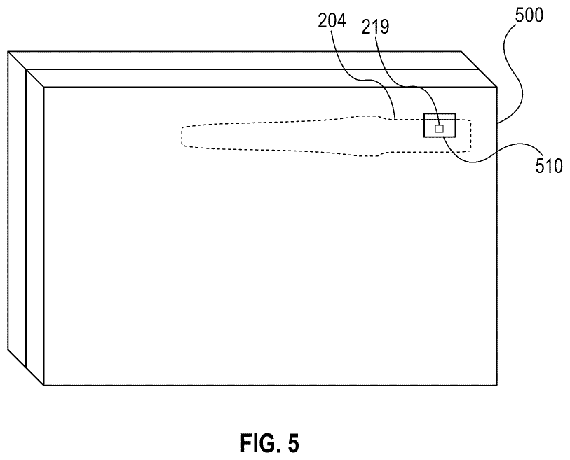

In some examples, indicators on handle 204 can be utilized to determine whether one or more actuators 208 have detached from frame 102 during transport. This can be determined before implantation of the device within a patient and/or at any point during the life cycle of prosthetic valve 100 and delivery apparatus 200. For example, as illustrated in FIG. 5, a case 500 can be provided, which contains prosthetic valve 100 and delivery apparatus 200 (not shown). In some examples, case 500 comprises a window 510. Window 510 is aligned with handle 204 such that indicator 219 can be seen via window 510. Thus, a detachment of one or more actuators 208 can be detected at any point in time.

In some examples (not shown), an indicator is provided on an external surface of case 500. In some examples (not shown), case 500 is transparent, thereby allowing indicator 219 to be seen at any point in time.

Although the above has been described in relation to an example where pressure is sensed at the distal end of central protrusion 306, this is not meant to be limiting in any way, and pressure can be measured at any point where actuator 208 is in contact with frame 102.

Although the above has been described in relation to a pressure sensor 320, this is not meant to be limiting in any way. In some examples (not shown), sensor 320 comprises an optical sensor which senses light being reflected from an external surface facing sensor 320. In such an example, detection module 218 identifies a change in the reflectance of the light, thereby identifying that actuator 208 has detached from frame 102. In some examples, one or more predetermined characteristics of the reflected light are compared to predetermined values, and based at least in part on the outcome of the comparison/s, detection module 218 determines the attachment status of actuator 208.

Thus, the above described examples for sensor 320 and detection module 218 provide a system for detecting the following: whether actuator 208 has fully detached from prosthetic valve 100; whether actuator 208 has partially detached from prosthetic valve 100; whether actuator 208 is fully attached to prosthetic valve 100; and/or whether actuator 208 is fully attached to prosthetic valve 100.

As described above, in some examples a plurality of actuators 208 are provided, each actuator 208 secured to a respective threaded rod 126. In such an example, each actuator 208 is provided with a respective sensor 320 and detection module 218 determines the attachment status of each actuator 208 is relation to prosthetic valve 100.

Although the above has been described in relation to prosthetic valve 100, this is not meant to be limiting in any way, and the above described system and method can similarly be used for prosthetic valve 100a, or a different type of prosthetic valve, as long as an actuator is pressed against the frame of the prosthetic valve.

### Some Examples of the Disclosed Technology

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve actuator attachment status detection system, comprising: at least one actuator; a frame of the prosthetic valve movable by the at least one actuator between a radially compressed configuration and a radially expanded configuration; and at least one pressure sensor secured to the at least one actuator.

Example 2. The system of any example herein, particularly example 1, wherein the at least one pressure sensor is an optical pressure sensor.

Example 3. The system of any example herein, particularly example 2, wherein the at least one pressure sensor is a fiber-optic pressure sensor.

Example 4. The system of any example herein, particularly any one of examples 1 to 3, wherein the at least one pressure sensor is embedded in the at least one actuator.

Example 5. The system of any example herein, particularly any one of examples 1 to 4, wherein the at least one pressure sensor is positioned at an edge of the at least one actuator.

Example 6. The system of any example herein, particularly any one of examples 1 to 4, wherein the at least one pressure sensor is position at a distal end of the at least one actuator.

Example 7. The system of any example herein, particularly any one of examples 1 to 6, further comprising a detection module configured to: receive an output of the at least one pressure sensor; based at least in part on the received output, determine the attachment status of the at least one actuator in relation to the frame; and based at least in part on the determination, output an indication of the determined attachment status.

Example 8. The system of any example herein, particularly example 7, wherein the determined attachment status comprises an indication of whether the at least one actuator has detached from the frame.

Example 9. The system of any example herein, particularly example 8, wherein the determined attachment status comprises an indication of whether the at least one actuator has fully detached from the frame.

Example 10. The system of any example herein, particularly any one of examples 7 to 9, wherein the determined attachment status is an indication of whether the at least one actuator is attached to the frame.

Example 11. The system of any example herein, particularly example 10, wherein the determined attachment status is an indication of whether the at least one actuator is correctly attached to the frame.

Example 12. The system of any example herein, particularly any one of examples 7 to 11, wherein the detection module is further configured to compare the received output of the at least one pressure sensor with a predetermined pressure value, the determination of the attachment status based at least in part on an outcome of the comparison.

Example 13. The system of any example herein, particularly any one of examples 7 to 12, wherein the frame comprises: at least one axially extending post, each of the at least one axially extending post comprising a proximal member and a distal member that are axially movable relative to one another to permit the frame to radially expand and/or compress, at least one rod, each associated with a respective one of the at least one axially extending post and rotatably coupled to the proximal member and distal member thereof, wherein a rotation of the at least one rod radially expands and/or radially compresses the frame, the rotation of the at least one rod performed by the at least one actuator, and wherein the determination of the attachment status of the at least one actuator comprises a determination of whether the at least one actuator is attached to the at least one rod.

Example 14. The system of any example herein, particularly example 13, wherein each of the proximal member and distal member of each of the at least one axially extending post comprise an inner bore, the respective one of the at least one rod extending through the inner bores of the proximal member and distal member.

Example 15. The system of any example herein, particularly example 13 or 14, wherein each of the at least one actuator comprises: a pair of elongated members; and a movable sleeve configured to compress the pair of elongated members such that the compressed pair of elongated members are fastened to a respective one of the at least one rod, wherein when the movable sleeve is moved away from the pair of elongated members the pair of elongated members detach from the respective rod, the determination of the attachment status of the at least one actuator comprising a determination whether the pair of elongated members have detached from the respective rod.

Example 16. The system of any example herein, particularly any one of examples 13 to 15, wherein each of the at least one actuator comprises: a pair of elongated members; and a movable sleeve configured to compress the pair of elongated members such that the of elongated members detach from the respective rod, the determination of the attachment status of the at least one actuator comprising a determination whether the pair of elongated members are attached to the respective rod.

Example 17. The system of any example herein, particularly any one of examples 13 to 16, wherein the at least one axially extending post comprises a plurality of axially extending posts and the at least one rod comprises a plurality of rods, wherein the at least one actuator comprises a plurality of actuators, the rotation of each of the plurality of rods performed by a respective one of the plurality of actuators, and wherein the determination of the attachment status of the at least one actuator comprises a determination of the attachment status of each of the plurality of actuators.

Example 18. The system any example herein, particularly example 17, wherein the determination of the attachment status of the at least one actuator comprises a determination whether each of the plurality of actuators has detached from the respective rod.

Example 19. The system any example herein, particularly example 18, wherein the determination of the attachment status of the at least one actuator comprises a determination whether each of the plurality of actuators has fully detached from the respective rod.

Example 20. The system of any example herein, particularly any one of examples 17 to 19, wherein the determination of the attachment status of the at least one actuator comprises a determination whether each of the plurality of actuators is attached to the respective rod.

Example 21. The system of any example herein, particularly example 20, wherein the determination of the attachment status of the at least one actuator comprises a determination whether each of the plurality of actuators is correctly attached to the respective rod.

Example 22. The system of any example herein, particularly any one of examples 7 to 21, further comprising an indicator configured to display the indication of the determined attachment status.

Example 23. The system of any example herein, particularly example 22, further comprising a case configured to secure the prosthetic valve therewithin, wherein the case comprises a window aligned with the indicator.

Example 24. The system of any example herein, particularly example 22, further comprising a case configured to secure the prosthetic valve therewithin, wherein the indicator is provided on an external surface of the case.

Example 25. The system of any example herein, particularly example 22, further comprising a case configured to secure the prosthetic valve therewithin, wherein the case is transparent.

Example 26. A prosthetic valve actuator attachment status detection system, comprising: at least one actuator; a frame of the prosthetic valve movable by the at least one actuator between a radially compressed configuration and a radially expanded configuration; and at least one optical sensor secured to the at least one actuator.

Example 27. The system of any example herein, particularly example 26, wherein the at least one optical sensor is a fiber-optic sensor.

Example 28. The system of any example herein, particularly example 26 or 27, wherein the at least one optical sensor is a pressure sensor.

Example 29. The system of any example herein, particularly any one of examples 26 to 28, wherein the at least one pressure sensor is embedded in the at least one actuator.

Example 30. The system of any example herein, particularly any one of examples 26 to 29, wherein the at least one pressure sensor is positioned at an edge of the at least one actuator.

Example 31. The system of any example herein, particularly any one of examples 26 to 29, wherein the at least one pressure sensor is position at a distal end of the at least one actuator.

Example 32. The system of any example herein, particularly any one of examples 26 to 31, further comprising a detection module configured to: receive an output of the at least one optical sensor; based at least in part on the received output, determine the attachment status of the at least one actuator in relation to the frame; and based at least in part on the determination, output an indication of the determined attachment status.

Example 33. The system of any example herein, particularly example 32, wherein the determined attachment status comprises an indication of whether the at least one actuator has detached from the frame.

Example 34. The system of any example herein, particularly example 33, wherein the determined attachment status comprises an indication of whether the at least one actuator has fully detached from the frame.

Example 35. The system of any example herein, particularly any one of examples 32 to 34, wherein the determined attachment status is an indication of whether the at least one actuator is attached to the frame.

Example 36. The system of any example herein, particularly example 35, wherein the determined attachment status is an indication of whether the at least one actuator is correctly attached to the frame.

Example 37. The system of any example herein, particularly any one of examples 32 to 36, wherein the detection module is further configured to compare the received output of the at least one optical sensor with a predetermined pressure value, the determination of the attachment status based at least in part on an outcome of the comparison.

Example 38. The system of any example herein, particularly any one of examples 32 to 37, wherein the frame comprises: at least one axially extending post, each of the at least one axially extending post comprising a proximal member and a distal member that are axially movable relative to one another to permit the frame to radially expand and/or compress, at least one rod, each associated with a respective one of the at least one axially extending post and rotatably coupled to the proximal member and distal member thereof, wherein a rotation of the at least one rod radially expands and/or radially compresses the frame, the rotation of the at least one rod performed by the at least one actuator, and wherein the determination of the attachment status of the at least one actuator comprises a determination of whether the at least one actuator is attached to the at least one rod.

Example 39. The system of any example herein, particularly example 38, wherein each of the proximal member and distal member of each of the at least one axially extending post comprise an inner bore, the respective one of the at least one rod extending through the inner bores of the proximal member and distal member.

Example 40. The system of any example herein, particularly example 38 or 39, wherein each of the at least one actuator comprises: a pair of elongated members; and a movable sleeve configured to compress the pair of elongated members such that the compressed pair of elongated members are fastened to a respective one of the at least one rod, wherein when the movable sleeve is moved away from the pair of elongated members the pair of elongated members detach from the respective rod, the determination of the attachment status of the at least one actuator comprising a determination whether the pair of elongated members have detached from the respective rod.

Example 41. The system of any example herein, particularly any one of examples 38 to 40, wherein each of the at least one actuator comprises: a pair of elongated members; and a movable sleeve configured to compress the pair of elongated members such that the of elongated members detach from the respective rod, the determination of the attachment status of the at least one actuator comprising a determination whether the pair of elongated members are attached to the respective rod.

Example 42. The system of any example herein, particularly any one of examples 38 to 41, wherein the at least one axially extending post comprises a plurality of axially extending posts and the at least one rod comprises a plurality of rods, wherein the at least one actuator comprises a plurality of actuators, the rotation of each of the plurality of rods performed by a respective one of the plurality of actuators, and wherein the determination of the attachment status of the at least one actuator comprises a determination of the attachment status of each of the plurality of actuators.

Example 43. The system any example herein, particularly example 42, wherein the determination of the attachment status of the at least one actuator comprises a determination whether each of the plurality of actuators has detached from the respective rod.

Example 44. The system any example herein, particularly example 43, wherein the determination of the attachment status of the at least one actuator comprises a determination whether each of the plurality of actuators has fully detached from the respective rod.

Example 45. The system of any example herein, particularly any one of examples 42 to 44, wherein the determination of the attachment status of the at least one actuator comprises a determination whether each of the plurality of actuators is attached to the respective rod.

Example 46. The system of any example herein, particularly example 45, wherein the determination of the attachment status of the at least one actuator comprises a determination whether each of the plurality of actuators is correctly attached to the respective rod.

Example 47. The system of any example herein, particularly any one of examples 32 to 46, further comprising an indicator configured to display the indication of the determined attachment status.

Example 48. The system of any example herein, particularly example 47, further comprising a case configured to secure the prosthetic valve therewithin, wherein the case comprises a window aligned with the indicator.

Example 49. The system of any example herein, particularly example 47, further comprising a case configured to secure the prosthetic valve therewithin, wherein the indicator is provided on an external surface of the case.

Example 50. The system of any example herein, particularly example 47, further comprising a case configured to secure the prosthetic valve therewithin, wherein the case is transparent.

Example 51. A prosthetic valve actuator attachment status detection method, the method comprising: sensing pressure applied between at least one actuator and a frame of the prosthetic valve movable by the at least one actuator between a radially compressed configuration and a radially expanded configuration; and outputting the sensed pressure.

Example 52. The method of any example herein, particularly example 51, wherein the sensing pressure is performed by an optical pressure sensor.

Example 53. The method of any example herein, particularly example 52, wherein the sensing pressure is performed by a fiber-optic pressure sensor.

Example 54. The method of any example herein, particularly any one of examples 51 to 53, wherein the at least one pressure sensor is embedded in the at least one actuator.

Example 55. The method of any example herein, particularly any one of examples 51 to 54, wherein the at least one pressure sensor is positioned at an edge of the at least one actuator.

Example 56. The method of any example herein, particularly any one of examples 51 to 54, wherein the at least one pressure sensor is position at a distal end of the at least one actuator.

Example 57. The method of any example herein, particularly any one of examples 51 to 56, further comprising: receiving an output of the at least one pressure sensor; based at least in part on the received output, determining the attachment status of the at least one actuator in relation to the frame; and based at least in part on the determination, outputting an indication of the determined attachment status.

Example 58. The method of any example herein, particularly example 57, wherein the determined attachment status comprises an indication of whether the at least one actuator has detached from the frame.

Example 59. The method of any example herein, particularly example 58, wherein the determined attachment status comprises an indication of whether the at least one actuator has fully detached from the frame.

Example 60. The method of any example herein, particularly any one of examples 57 to 59, wherein the determined attachment status is an indication of whether the at least one actuator is attached to the frame.

Example 61. The method of any example herein, particularly example 60, wherein the determined attachment status is an indication of whether the at least one actuator is correctly attached to the frame.

Example 62. The method of any example herein, particularly any one of examples 57 to 61, further comprising comparing the received output of the at least one pressure sensor with a predetermined pressure value, the determination of the attachment status based at least in part on an outcome of the comparison.

Example 63. The method of any example herein, particularly any one of examples 57 to 61, wherein the frame comprises: at least one axially extending post, each of the at least one axially extending post comprising a proximal member and a distal member that are axially movable relative to one another to permit the frame to radially expand and/or compress, at least one rod, each associated with a respective one of the at least one axially extending post and rotatably coupled to the proximal member and distal member thereof, wherein a rotation of the at least one rod radially expands and/or radially compresses the frame, the rotation of the at least one rod performed by the at least one actuator, and wherein the determination of the attachment status of the at least one actuator comprises a determination of whether the at least one actuator is attached to the at least one rod.

Example 64. The method of any example herein, particularly example 63, wherein each of the proximal member and distal member of each of the at least one axially extending post comprise an inner bore, the respective one of the at least one rod extending through the inner bores of the proximal member and distal member.

Example 65. The method of any example herein, particularly example 63 or 64, wherein each of the at least one actuator comprises: a pair of elongated members; and a movable sleeve configured to compress the pair of elongated members such that the compressed pair of elongated members are fastened to a respective one of the at least one rod, wherein when the movable sleeve is moved away from the pair of elongated members the pair of elongated members detach from the respective rod, the determination of the attachment status of the at least one actuator comprising a determination whether the pair of elongated members have detached from the respective rod.

Example 66. The method of any example herein, particularly any one of examples 63 to 65, wherein each of the at least one actuator comprises: a pair of elongated members; and a movable sleeve configured to compress the pair of elongated members such that the of elongated members detach from the respective rod, the determination of the attachment status of the at least one actuator comprising a determination whether the pair of elongated members are attached to the respective rod.

Example 67. The method of any example herein, particularly any one of examples 63 to 66, wherein the at least one axially extending post comprises a plurality of axially extending posts and the at least one rod comprises a plurality of rods, wherein the at least one actuator comprises a plurality of actuators, the rotation of each of the plurality of rods performed by a respective one of the plurality of actuators, and wherein the determination of the attachment status of the at least one actuator comprises a determination of the attachment status of each of the plurality of actuators.

Example 68. The method any example herein, particularly example 67, wherein the determination of the attachment status of the at least one actuator comprises a determination whether each of the plurality of actuators has detached from the respective rod.

Example 69. The method any example herein, particularly example 68, wherein the determination of the attachment status of the at least one actuator comprises a determination whether each of the plurality of actuators has fully detached from the respective rod.

Example 70. The method of any example herein, particularly any one of examples 67 to 69, wherein the determination of the attachment status of the at least one actuator comprises a determination whether each of the plurality of actuators is attached to the respective rod.

Example 71. The method of any example herein, particularly example 70, wherein the determination of the attachment status of the at least one actuator comprises a determination whether each of the plurality of actuators is correctly attached to the respective rod.

Example 72. A prosthetic valve actuator attachment status detection method, the method comprising receiving optical sensor data from at least one optical sensor secured to at least one actuator, wherein a frame of the prosthetic valve is movable by the at least one actuator between a radially compressed configuration and a radially expanded configuration.

Example 73. The method of any example herein, particularly example 72, wherein the at least one optical sensor is a fiber-optic sensor.

Example 74. The method of any example herein, particularly example 72 or 73, wherein the at least one optical sensor is a pressure sensor.

Example 75. The method of any example herein, particularly any one of examples 72 to 74, wherein the at least one pressure sensor is embedded in the at least one actuator.

Example 76. The method of any example herein, particularly any one of examples 72 to 75, wherein the at least one pressure sensor is positioned at an edge of the at least one actuator.

Example 77. The method of any example herein, particularly any one of examples 72 to 75, wherein the at least one pressure sensor is position at a distal end of the at least one actuator.

Example 78. The method of any example herein, particularly any one of examples 72 to 77, further comprising: based at least in part on the received optical sensor data, determining the attachment status of the at least one actuator in relation to the frame; and based at least in part on the determination, outputting an indication of the determined attachment status.

Example 79. The method of any example herein, particularly example 78, wherein the determined attachment status comprises an indication of whether the at least one actuator has detached from the frame.

Example 80. The method of any example herein, particularly example 79, wherein the determined attachment status comprises an indication of whether the at least one actuator has fully detached from the frame.

Example 81. The method of any example herein, particularly any one of examples 78 to 80, wherein the determined attachment status is an indication of whether the at least one actuator is attached to the frame.

Example 82. The method of any example herein, particularly example 81, wherein the determined attachment status is an indication of whether the at least one actuator is correctly attached to the frame.

Example 83. The method of any example herein, particularly any one of examples 78 to 82, further comprising comparing the received optical sensor data with a predetermined pressure value, the determination of the attachment status based at least in part on an outcome of the comparison.

Example 84. The method of any example herein, particularly any one of examples 78 to 83, wherein the frame comprises: at least one axially extending post, each of the at least one axially extending post comprising a proximal member and a distal member that are axially movable relative to one another to permit the frame to radially expand and/or compress, at least one rod, each associated with a respective one of the at least one axially extending post and rotatably coupled to the proximal member and distal member thereof, wherein a rotation of the at least one rod radially expands and/or radially compresses the frame, the rotation of the at least one rod performed by the at least one actuator, and wherein the determination of the attachment status of the at least one actuator comprises a determination of whether the at least one actuator is attached to the at least one rod.

Example 85. The method of any example herein, particularly example 84, wherein each of the proximal member and distal member of each of the at least one axially extending post comprise an inner bore, the respective one of the at least one rod extending through the inner bores of the proximal member and distal member.

Example 86. The method of any example herein, particularly example 84 or 85, wherein each of the at least one actuator comprises: a pair of elongated members; and a movable sleeve configured to compress the pair of elongated members such that the compressed pair of elongated members are fastened to a respective one of the at least one rod, wherein when the movable sleeve is moved away from the pair of elongated members the pair of elongated members detach from the respective rod, the determination of the attachment status of the at least one actuator comprising a determination whether the pair of elongated members have detached from the respective rod.

Example 87. The method of any example herein, particularly any one of examples 84 to 86, wherein each of the at least one actuator comprises: a pair of elongated members; and a movable sleeve configured to compress the pair of elongated members such that the compressed pair of elongated members are fastened to a respective one of the at least one rod, wherein when the movable sleeve is moved away from the pair of elongated members the pair of elongated members detach from the respective rod, the determination of the attachment status of the at least one actuator comprising a determination whether the pair of elongated members are attached to the respective rod.

Example 88. The method of any example herein, particularly any one of examples 84 to 87, wherein the at least one axially extending post comprises a plurality of axially extending posts and the at least one rod comprises a plurality of rods, wherein the at least one actuator comprises a plurality of actuators, the rotation of each of the plurality of rods performed by a respective one of the plurality of actuators, and wherein the determination of the attachment status of the at least one actuator comprises a determination of the attachment status of each of the plurality of actuators.

Example 89. The method any example herein, particularly example 88, wherein the determination of the attachment status of the at least one actuator comprises a determination whether each of the plurality of actuators has detached from the respective rod.

Example 90. The method any example herein, particularly example 89, wherein the determination of the attachment status of the at least one actuator comprises a determination whether each of the plurality of actuators has fully detached from the respective rod.

Example 91. The method of any example herein, particularly any one of examples 88 to 90, wherein the determination of the attachment status of the at least one actuator comprises a determination whether each of the plurality of actuators is attached to the respective rod.

Example 92. The method of any example herein, particularly example 91, wherein the determination of the attachment status of the at least one actuator comprises a determination whether each of the plurality of actuators is correctly attached to the respective rod.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

We claim:

1. A prosthetic valve actuator attachment status detection system, comprising:
at least one actuator;
a frame of the prosthetic valve movable by the at least one actuator between a radially compressed configuration and a radially expanded configuration; and
at least one pressure sensor secured to the at least one actuator.

2. The system of claim 1, wherein the at least one pressure sensor is an optical pressure sensor.

3. The system of claim 2, wherein the at least one pressure sensor is a fiber-optic pressure sensor.

4. The system of claim 1, wherein the at least one pressure sensor is embedded in the at least one actuator.

5. The system of claim 1, wherein the at least one pressure sensor is positioned at an edge of the at least one actuator.

6. The system of claim 1, further comprising a detection module configured to:
receive an output of the at least one pressure sensor;
based at least in part on the received output, determine the attachment status of the at least one actuator in relation to the frame; and
based at least in part on the attachment status, output an indication of the determined attachment status.

7. The system of claim 6, wherein the determined attachment status comprises an indication of whether the at least one actuator has detached from the frame.

8. The system of claim 6, wherein the determined attachment status is an indication of whether the at least one actuator is attached to the frame.

9. The system of claim 8, wherein the determined attachment status is an indication of whether the at least one actuator is correctly attached to the frame.

10. The system of claim 6, wherein the detection module is further configured to compare the received output of the at least one pressure sensor to a predetermined pressure value, wherein the determination of the attachment status is based at least in part on an outcome of the comparison.

11. The system of claim 6, wherein the frame comprises: at least one axially extending post, each of the at least one axially extending post comprising a proximal member and a distal member that are axially movable relative to one another to permit the frame to radially expand and/or compress,
at least one rod, each associated with a respective one of the at least one axially extending post and rotatably coupled to the proximal member and distal member thereof,
wherein a rotation of the at least one rod radially expands and/or radially compresses the frame, the rotation of the at least one rod performed by the at least one actuator, and
wherein the determination of the attachment status of the at least one actuator comprises a determination of whether the at least one actuator is attached to the at least one rod.

12. The system of claim 11, wherein each of the proximal member and distal member of each of the at least one axially extending post comprise an inner bore, the respective one of the at least one rod extending through the inner bores of the proximal member and distal member.

13. The system of claim 11, wherein each of the at least one actuator comprises:
a pair of elongated members; and
a movable sleeve configured to compress the pair of elongated members such that the compressed pair of elongated members are fastened to a respective one of the at least one rod,
wherein when the movable sleeve is moved away from the pair of elongated members the pair of elongated members detach from the respective rod, the determination of the attachment status of the at least one actuator comprising a determination whether the pair of elongated members have detached from the respective rod.

14. The system of claim 11, wherein each of the at least one actuator comprises:
a pair of elongated members; and
a movable sleeve configured to compress the pair of elongated members such that the compressed pair of elongated members are fastened to a respective one of the at least one rod,
wherein when the movable sleeve is moved away from the pair of elongated members the pair of elongated members detach from the respective rod, the determination of the attachment status of the at least one actuator comprising a determination whether the pair of elongated members are attached to the respective rod.

15. The system of claim 11, wherein the at least one axially extending post comprises a plurality of axially extending posts and the at least one rod comprises a plurality of rods,
wherein the at least one actuator comprises a plurality of actuators, the rotation of each of the plurality of rods performed by a respective one of the plurality of actuators, and
wherein the determination of the attachment status of the at least one actuator comprises a determination of the attachment status of each of the plurality of actuators.

16. The system of claim 15, wherein the determination of the attachment status of the at least one actuator comprises a determination whether each of the plurality of actuators has detached from the respective rod.

17. The system of claim 15, wherein the determination of the attachment status of the at least one actuator comprises a determination whether each of the plurality of actuators is attached to the respective rod.

18. The system of claim 17, wherein the determination of the attachment status of the at least one actuator comprises a determination whether each of the plurality of actuators is correctly attached to the respective rod.
